(19) United States
(12) Patent Application Publication
(10) Pub. No.: US 2004/0247594 A1
(43) Pub. Date: Dec. 9, 2004

(54) Use of cd28-specific monoclonal antibodies for stimulating blood cells that lack cd28

(76) Inventors: Thomas Hunig, Wurzberg (DE); Marta Rodriguez-Palmero, Munchen (DE); Thomas Kerkau, Wurzberg (DE)

Correspondence Address: Mark D Wieczorek, PO Box 70072, San Diego, CA, 92167, US

(21) Appl. No.: 10/399,056

(22) PCT Filed: Sep. 28, 2001
(86) PCT No.: PCT/DE01/03802

(30) Foreign Application Priority Data
Oct. 11, 2000 (DE) 100-50-935.5

(51) Int. Cl. A61K 39/395; C07K 16/28

(52) U.S. Cl. 424/144.1; 530/388.22

(57) ABSTRACT

The invention teaches the use of monoclonal antibodies being specific for CD28 and activating T lymphocytes of several to all sub-groups without an occupation of an antigen receptor of the T lymphocytes and thus in an antigen-unspecific manner, or of an analogue hereto, for the preparation of a pharmaceutical composition for stimulating blood cells not carrying CD28, and for treating diseases with a reduced number of such blood cells.

### FIELD OF THE INVENTION

The invention relates to a use of CD28-specific monoclonal antibodies that are specific for CD28 and that activate the T lymphocytes without occupying an antigen receptor of the T lymphocytes and thus in an antigen-unspecific manner.

### BACKGROUND OF THE INVENTION

There exist various diseases for warm-blooded organisms, human or animal, wherein the number of different blood cells is reduced in comparison to the healthy condition.

A first such group of diseases is called granulocytopenia (neutropenia and monocytopenia) and relates to the granulocytes. Causes for this disease are: i) reduced granulocytopoiesis (aplastic disturbance) due to bone marrow damages, for instance by chemicals such as benzol, drugs such as cytostatics, immuno-suppressives, AZT and/or chloramphenicol (dose-dependent, toxic) or phenylbutazon, gold compounds, rarely chloramphenicol (dose-independent by pharmacogenetic reactions), rays or autoantibodies against stem cells (in some cases of immunoneutropenia), because of bone marrow infiltration (leukaemiae, carcinomas, malignant lymphomas) and/or because of osteomyelosclerosis, ii) maturation disturbances of the granulocytopoiesis, for instance congenital maturation disturbances of the myelopoiesis, Kostmann syndrome (maturation arrest of the myelopoiesis at the stage of the promyelocyte), cyclic neutropenia, myelodysplasia syndrome, vitamin B12 or folic acid deficiency with ineffective granulo, erythro and/or thrombopoiesis. Usual therapies include the administration of growth factors of the granulopoiesis (for instance G-CSF and GM-CSF).

A second group is called thrombocytopenia. Causes may be: i) reduced thrombocytopoiesis in the bone marrow (aplastic disturbance or reduced number of megakaryocytes in the bone marrow) due to bone marrow damages, for instance by chemicals such as benzol, drugs such as cytostatics, immuno-suppressives, rays, infections such as HIV, or autoantibodies against megakaryocytes (in some cases of immunothrombocytopenia), because of bone marrow infiltration (leukaemiae, carcinomas, malignant lymphomas) and/or because of osteomyelosclerosis, ii) maturation disturbances of the megakaryocytes (megakaryocytes in bone marrow normal or increased) with ineffective thrombo, erythro and/or granulopoiesis with megaloblasts, giant rods and others because of vitamin B12 or folic acid deficiency. Usual therapies include the omission of suspicious drugs, thrombocyte substitution (in case of disturbances in the bone marrow: thrombopoietin) as well as MGDF (stimulation of the proliferation and maturation of megakaryocytes).

A third group are the aplastic anemias or bone marrow failures with aplasia/hypoplasia of the bone marrow and pancytopenia (stem cell disease). An inherited aplastic anemia is for instance the Fanconi anemia. More frequently occur the acquired aplastic anemias, such as the idiopathic aplastic anemia (cause unknown) and the secondary aplastic anemia by drugs, toxic substances, ionizing radiations and virus infections (see above). Supportive therapy approaches include the substitution of erythrocytes/thrombocytes. Causal therapy approaches include the bone marrow transplantation or stem cells transplantation, immuno-suppressive therapies (such as ATG) and other therapy measures, such as administration of cytokines (GM-CSF, GCSF, MGDF and/or thrombopoietin).

Finally, the acute leukemia often occurs as an anemia, thrombocytopenia and/or granulocytopenia. Therapies include the substitution of erythrocytes and thrombocytes according to requirements or the excitation of the granulopoiesis by G-CSF and/or GM-CSF, the chemotherapy and the bone marrow and/or stem cells transplantation.

It is common to the above diseases that the concerned blood cells are those which do not carry CD28 on their surface.

### PRIOR ART

CD28 is a cell surface molecule of a known amino acid sequence expressed on T lymphocytes of human or animal origin, this molecule having obtained the abbreviation CD28 by the international “Human Leukocyte Typing Workshop”. An activation of T lymphocytes is the multiplication of the metabolism activity, enlargement of the cell volume, synthesis of immunologically important molecules and beginning of the cell division (proliferation) of T lymphocytes upon an external stimulation. These processes are initiated for instance by the occupation of the CD28 molecule on T cells by special CD28-specific monoclonal antibodies. The activation of the T lymphocytes with the described side effects is part of the physiological immune reaction, may however be lost out of control there in pathological situations (lymphoproliferative diseases) or may be insufficient (immune deficiency).

For understanding the invention, firstly the following terminological background is important. The activation of resting T cells for the proliferation and functional differentiation requires the occupation of two surface structures, so-called receptors: 1, of the antigen receptor having a different specificity from cell to cell and being necessary for detecting antigens, for instance viral fission products; and of the CD28 molecule equally expressing on all resting T cells, the CD28 molecule naturally binding to ligands on the surface of other cells of the immune system. It is the “costimulation” of the antigen-specific immune reaction by CD28. In a cell culture, these processes can be imitated by occupation of the antigen receptor and of the CD28 molecule with suitable monoclonal antibodies. In the classic system of the costimulation, neither the occupation of the antigen receptor nor that of the CD28 alone will lead to a cell proliferation, the occupation of both receptors is however effective. This observation was made at T cells of man, mouse and rat.

A “direct”, i.e. independent from the occupation of the antigen receptor, activation of resting T lymphocytes by CD28-specific monoclonal antibodies has been observed in the following systems: in the document Brinkmann et al., J. Immunology, 1996, 156:4100-4106 it has been shown that a very small share (5%) of human T lymphocytes carrying the surface marker CD45 RO being typical for resting T lymphocytes are activated by the “classic” CD28-specific monoclonal antibody 9.3 upon addition of the growth factor interleukin-2 (IL-2) without an occupation of the antigen receptor. In the publication of Siefken et al., Cellular Immunology, 1997, 176:59-65, it has been shown that a CD28-specific monoclonal antibody prepared in a conventional way, i.e. by immunization, can activate in a cell culture a sub-group of human T cells without occupation of the antigen receptor for the proliferation, if CD28 is occupied by this monoclonal antibody and the cell-bound monoclonal antibody molecules are in addition crosslinked with one another by further antibodies. In either case, the described antibodies are originally in principle not suitable for use in human medicine, since these are mouse antibodies. Further it is common to both described antibodies that a small share only of the T cells can “directly” be activated.

In the publication of Tacke et al., Eur. J. Immunol., 1997, 27:239-247, two types of CD28-specific monoclonal antibodies having different functional properties have been described: “classic antibodies” costimulating the activation of resting T cells only with simultaneous occupation of the antigen receptor; and “direct antibodies” being able to activate without occupation of the antigen receptor T lymphocytes of all classes for proliferation in vitro and in a test animal. Both in so far known monoclonal antibodies originate from an immunization with cells on which rat CD28 is expressed, and are obtainable by different selections determined by their respectively described properties.

From the document WO 98/54225 to which explicitly reference is made in order to avoid repetitions, “direct” CD28-specific monoclonal antibodies are known in the art, same as the use thereof for treating diseases with pathologically reduced CD4 T cell counts or for modulating immune reactions in the case of vaccinations. In these treatments, cells are concerned which carry CD28 on their surface (T cells), and consequently are immediately stimulated by the monoclonal antibodies.

From the unpublished patent application DE 199 39 653, it is known to use “direct” CD28-specific monoclonal antibodies together with virus inhibitors for viral infections of T lymphocytes. Here, too, therefore cells are concerned which carry CD28 on their surface, and consequently are immediately stimulated by the monoclonal antibodies.

### TECHNICAL OBJECT OF THE INVENTION

The invention is based on the technical object to specify a pharmaceutical composition, by means of which the generation and/or activation of blood cells not carrying CD28 is stimulated. Basics of the invention.

For achieving the above technical object, the invention teaches the use of monoclonal antibodies being specific for CD28 and activating T lymphocytes of several to all sub-groups without an occupation of an antigen receptor of the T lymphocytes and thus in an antigen-unspecific manner, or of an analogue hereto, for the preparation of a pharmaceutical composition for stimulating blood cells not carrying CD28, and the use of monoclonal antibodies being specific for CD28 and activating T lymphocytes of several to all sub-groups without an occupation of an antigen receptor of the T lymphocytes and thus in an antigen-unspecific manner, or of an analogue hereto, for the preparation of a pharmaceutical composition for treating diseases with a reduced number of blood cells not carrying CD28. The blood cells may be granulocytes, monocytes and/or thrombocytes. In the case of diseases, these may in particular be the above-mentioned diseases.

Stimulation is the multiplication of the metabolism activity, enlargement of the cell volume, synthesis of factors and/or beginning of the proliferation.

Analogues are substances not being monoclonal antibodies, however fulfilling the functions as described in this invention. Examples are “tailored” highly specific synthetic proteins or RNA, DNA or PNA (for instance aptamers, in particular aptamers stabilized against nucleic acid-splitting enzymes or RNA, DNA or PNA). A common criterion always is the CD28 specificity with a “directly” stimulatory effect for CD28-carrying blood cells.

The invention relies on the surprising detection that the CD28-specific monoclonal antibodies also cause the proliferation or generation or activation of blood cells not carrying CD28, i.e. blood cells wherein a coupling of the antibodies not seems to be possible. Without being bound to a theory, it is assumed that the surprising effect relies on that by means of the “direct” stimulation (i.e. without a costimulant) of CD28-carrying cells, in particular T, cells, these cells in turn produce and release factors then again stimulating the blood cells not carrying CD28. In so far, this is presumably an indirect process.

For pharmaceutical compositions administered to human beings, the monoclonal antibodies according to the invention preferably comprise human constant components. Constant components of an antibody are regions which are not significant for the antigen detection, in contrast to the variable regions defining the antigen specificity of an antibody. Constant components are however different in antibodies of different types, and thus also for animal and man. The constant regions of an antibody may for instance correspond to those of antibodies of an organism to be treated with antibodies in order to be tolerable. Monoclonal antibodies used according to the invention in man are thus on the one hand well tolerated by man, per se or by humanization, and may on the other hand serve for the treatment, since the antibodies may be adapted as specific against human-CD28, and since the activation of the T lymphocytes is comprehensive. With a corresponding adaptation or preparation of a derivative, monoclonal antibodies used according to the invention can also be employed for non-human mammals. It should be noted here that non-humanized monoclonal antibodies may nevertheless be well tolerated by man. Well tolerated by man are monoclonal antibodies, if they will not cause undesired immune reactions for a certain period of time, as detectable for instance by the determination of anti-immunoglobulin antibodies, if these prohibit their application.

The invention also covers different derivatives of the monoclonal antibodies according to the invention, if the claimed features are fulfilled. Derivatives of monoclonal antibodies are modifications of the monoclonal antibodies produced by usual biochemical or gene-technological manipulations. This is for instance the case for the humanization of a monoclonal antibody of the mouse by partial replacement of structural (constant) components of the mouse antibody by those of a human one.

In detail, the monoclonal antibodies used according to the invention are obtainable by: A) preparation of hybridoma cells suitable to produce monoclonal human CD28-specific animal antibodies in the course of an immunization with non-T tumor cell lines on which human CD28 is expressed, or with recombinantly expressed CD28, B) if applicable, humanization of the monoclonal animal antibodies obtainable from the hybridoma cells according to step A by biochemical or gene-technological replacement of constant components of the animal antibodies by analogous constant components of a human antibody or replacement of the components of corresponding genes of the hybridoma cells, C) secernment of the antibodies in hybridoma cultures and isolation of the antibodies or production of the antibodies by injection of the hybridoma cells in animals, for instance mice, and isolation of the antibodies from the body liquid of the animals.

An important aspect of the monoclonal antibodies used according to the invention, with regard to the closest documents Brinkmann et al., J. Immunology, 1996, 156:4100-4106, and Siefken et al., Cellular Immunology, 1997, 176:59-65, is therefore the finding that the monoclonal antibodies are obtained by immunization with non-T tumor cells on which human CD28 is expressed, in lieu of an immunization with T cell lines. For, thereby can be obtained monoclonal antibodies which are not only specific for (human) CD28, but also effect a “direct” activation to a considerable extent. In detail, monoclonal antibodies used according to the invention can have specificity for determinants for instance of the human CD28 molecule, which are difficultly accessible on the naturally expressing CD28 molecule, and the occupation of which by the new monoclonal antibodies will lead to the activation of the T cells and in the end to the activation of blood cells not carrying CD28. A determinant is the region of a molecule which is defined by the binding specificity of one or more antibodies.

The basic approach for the preparation of hybridoma cells, the humanization and production of the monoclonal antibodies from the (humanized) hybridoma cells is well known to the man skilled in the art and needs not be explained here in detail. In principle, all cell lines being usual, known and freely accessible in particular for the preparation of the hybridoma cells can be used. For preparing the monoclonal antibodies, in principle, in addition to the approach described in the following, the recombinant expression being well known to the man skilled in the art can be used.

In detail, it is preferred if the hybridoma cells suitable for the production of monoclonal human CD28-specific animal antibodies are obtainable by a) provision of a plasmid by insertion of human CD28 cDNA into the pHβAPr-1 neo vector after excision of the SalI-HindIII fragment and preparation of protoplasts from Escherichia coli (MC1061) carrying the plasmid, b) fusion of the protoplasts with mouse A20J and/or L929 tumor cells by means of polyethylene glycol, c) cultivation of the cells obtained in step b, d) screening of the transfected mouse A20J and/or L929 cells on the expression of human CD28 and selection of human CD28 expressing mouse A20J and/or L929 cells, e) immunization of BALB/c nice with the human CD28 expressing mouse A20J and/or L929 cells (for instance by injections 6 times IP and once IV, f) removal of spleen cells of the thus immunized mice and fusion of the spleen cells with (“non-producer”, i.e. not producing antibodies) cells of the cell line X63-Ag 8.653 by means of polyethylene glycol, g) selection of the thus obtained hybridoma cells such that in the supernatant of selected hybridoma cells there are antibodies binding to human CD28 expressing mouse A20J and/or L929 cells, and h) cultivation/subcloning of the selected hybridoma cells obtained in step g. In lieu of steps a) to d), of course other expression systems known to the man skilled in the art may be used. Human CD28 cDNA is freely obtainable from Dr. A. Aruffo and Dr. B. Seed who have published the sequence and also the following document: Aruffo, A., and Seed, B., 1987, “Molecular cloning of CD28 cDNA by a high efficiency COS cell expression system”, Proc. Natl. Acad. Sci. USA, 84:8573. From this document can therefore be taken details of the preparation of the human CD28 cDNA. Furthermore, every man skilled in the art can very easily and quickly produce a human CD28 cDNA clone by means of the sequence filed in the gene library and the polymerase chain reaction. The pHβAPr-1-neo vector is freely obtainable from the authors of the document Gunning, P., et al., 1987, “A human β-actin expression vector system directs high-level accumulation of antisense transcripts”, Proc. Natl. Acad. Sci. USA, 84:4831. “neo” is here the neomycin resistance. The step c) is performed in presence of neomycin. The abovementioned cell lines and/or microorganism are freely accessible and commercially available from the American Type Culture Collection (ATCC). With regard to Escherichia coli (MC1061), reference is made to the document Meissner, P. S., et al., 1987, “Bacteriophage gamma cloning system for the construction of directional cDNA libraries”, Proc. Natl. Acad. Sci. USA, 84:4171.

The galenic preparation of the monoclonal antibodies used according to the invention for the various administration types is well known to the man skilled in the art and needs not be explained here in detail. In particular, in addition to the drug component according to the invention, further drugs and/or substances suitable or necessary for the galenic preparation may be included. It is understood that a pharmaceutical composition according to the invention contains the monoclonal antibodies in a therapeutically effective dose. A therapeutically active dose can be determined for the most various organisms by that the dose is determined which leads in a statistically significant manner to an increase of the number of blood cells not carrying CD28 in comparison to the situation prior to the administration of the composition.

The invention also comprises a method for treating the above diseases under application of monoclonal antibodies according to the invention.

In the following, the invention is explained in more detail, based on examples of execution.

The represented experiments or examples with regard to the effects of “direct” CD28-specific monoclonal antibodies were performed in the animal model of the rat, as an example for a “classic” CD28-specific antibody, the monoclonal antibody JJ319, and as an example for a “directly” activating one, the monoclonal antibody JJ316 being used. Both antibodies are freely accessible and commercially available from the company Pharmingen, San Diego, USA. JJ319 and JJ316 antibodies are further available according to the document M. Tacke et al., Immunology, 1995, 154:5121-5127, to which reference is explicitly made here, also with regard to details of the preparation of hybridoma cells and monoclonal antibodies.

### EXAMPLE 1

### Preparing CD28-Specific “Direct” Monoclonal Antibodies

In this example, the preparation of monoclonal antibodies used according to the invention, i.e. being human CD28 specific, is explained in more detail. These monoclonal antibodies are in the following also called CMY-2. Human CD28 from a cDNA library was recombinantly expressed in A20J and/or L929 cell lines. Firstly, a plasmid was prepared by means of insertion of human CD28 cDNA into the pHβAPr vector after excision of the SalL-HindIII fragment. From Escherichia coli (MC1061) were produced protoplasts carrying the plasmid. Then a fusion of the protoplasts was made with mouse A20J and/or L929 tumor cells by means of polyethylene glycol. The thus obtained transfected cells were cultivated in a usual way. Subsequently, a screening of the transfected mouse A20J and/or L929 cells on the expression of human CD28 and selection of human CD28 expressing mouse A20J and/or L929 cells was performed.

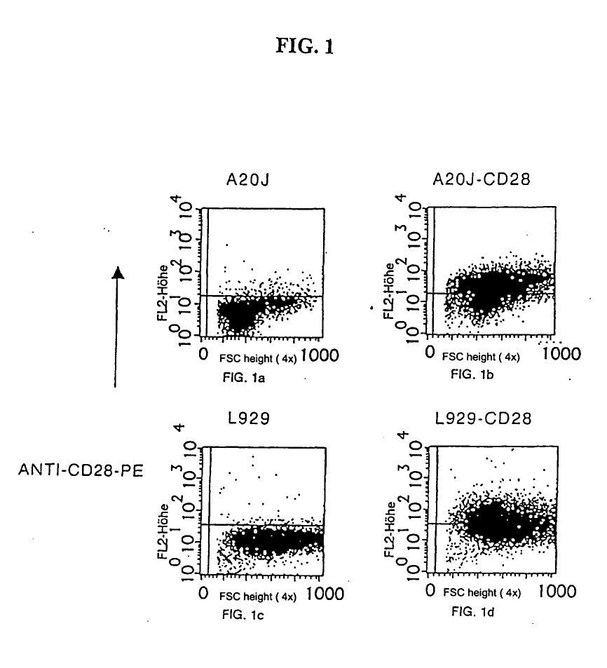

The detection of the successful expression was made by means of a conventional commercially available fluorescence-marked antibody with specificity for human CD28 (9.3-phycoerythrin). As a negative check, not transfected mouse A20J and/or L929 cells were dyed with the same antibody. The transfectants (A20J-CD28 and L929-CD28) showed a higher fluorescence intensity. Since not all cells were CD28-positive, CD28-positive cells were subcloned and used for the immunization. As can be seen in FIG. 1 from the displacement of the dot clouds towards top in the two right-hand diagrams, these cells reacted with the commercial antibody, i.e. expressed human CD28 on their surface.

The A20J human CD28 cell line was used for the immunization of BALB/c mice. Cell fusion and screening were performed as follows: i) Immunization of BALB/c mice with the human CD28 expressing mouse A20J cells (injections 6 times IP and then once IV), ii) Removal of spleen cells of the thus immunized mice and fusion of the spleen cells with cells of the cell line X63-Ag 8.653 by means of polyethylene glycol, iii) Selection of the thus obtained hybridoma cells such that in the supernatant of selected hybridoma cells there are antibodies binding to human CD28 expressing mouse A20J and/or L929 cells.

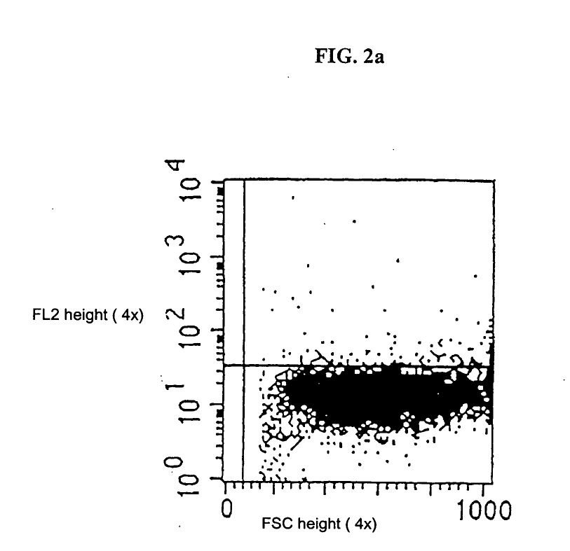

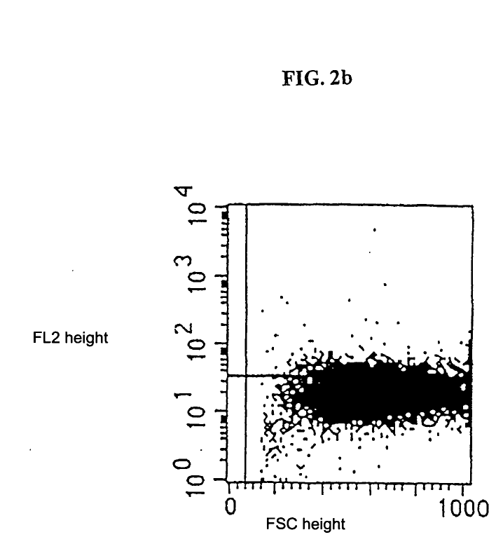

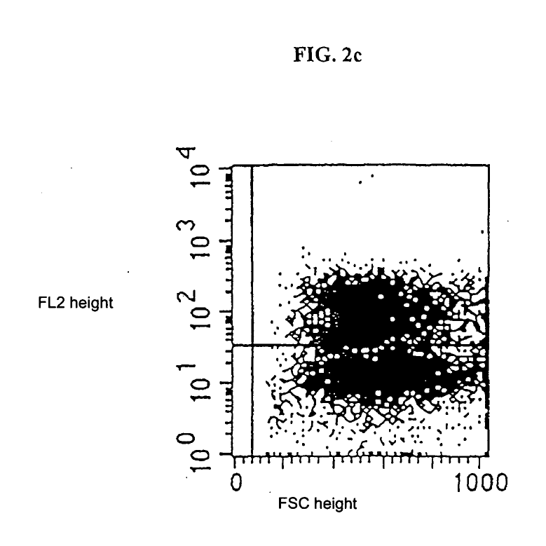

As a read-out served the coloration of a mixture of CD28-transfected and untransfected mouse L929 tumor cells. FIG. 2 shows that the monoclonal antibody CMY-2 isolated in this way distinguishes transfected and untransfected cells by different fluorescence intensity. The differential screening for antibodies against human CD28 was performed as follows. 50 μl supernatant each of cultivated hybridoma cells were taken out and incubated for 15 min. After washing, the cells were dyed with DaMIg-PE. Part A shows the negative check. The cells were incubated with DaMIg-PE only. Part B shows the coloration with a supernatant which was slightly positive, not showing however a difference for these two cells. Part C shows the cells dyed with a supernatant of CMY-2.

In not shown experiments, peripheral blood cells of man were dyed with the newly isolated CMY-2 and the “classic” CD28-specific antibody 9.3. An identical expression pattern was found on the subpopulations of human blood cells.

As a whole, the experiments showed that CMY-2 is a human CD28-specific antibody.

CMY-2 was then tested with human T lymphocytes enriched to approx. 80% from peripheral blood for classically costimulating and for “directly” stimulating activity. The T cell proliferation was measured by incorporation of 3H thymidine between the 2nd and 3rd day of the culture. The following results were obtained:

Costimulation:

| | |
|---|---|
| Uninstalled cells | 276 cpm |
| CD3-specific antibodies | 3,111 cpm |
| CD3-specific antibodies + CMY-2 | 51,676 cpm |

Direct Stimulation:

| | |
|---|---|
| Soldi-phase anti-mouse Ig | 379 cpm |
| Solid-phase anti-mouse Ig + control mAb | 258 cpm |
| Solid-phase anti-mouse Ig + CMY-2 | 19,115 cpm |

For a better understanding: anti-CD3 serves for T cell receptor stimulation (CD3 is a part of the TCR complex). CMY-2 was used in the form of a not purified culture supernatant (50% final volume). According to acquired experiences, the effective mAb concentration to be expected is sub-optimum for a direct activation, however sufficient for the costimulation. The experiment shows that CMY-2 has directly activating properties.

Hybridoma cells according to the invention producing CMY-2 have been filed at the DSMZ Deutsche Sammlung von Mikroorganismen und Zellkulturen GmbH, (German Collection of Microorganisms and Cell Cultures), Mascheroder Weg 1b, D-38124 Braunschweig, under number DSM ACC2353 (20 May 1998).

### EXAMPLE 3

### Reconstituting the T Cell Count in T Lymphopenic Rats with Mitogenic Anti-CD28 mAb

Treatment with mitogenic, not however with conventional anti-CD28 mAb will lead to a rapid reconstitution of the T cell counts in T lymphopenic rats, as will be shown hereafter.

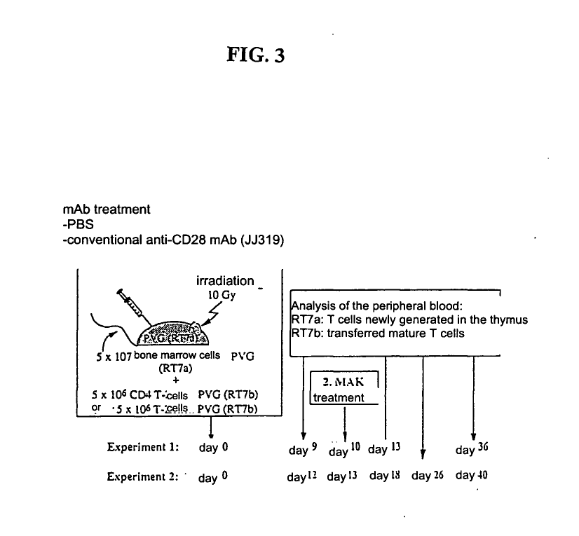

The test plan is shown in FIG. 3. PVG inbred rats were irradiated with gamma radiation of a cesium source so that the blood-forming system including the white blood cells is destroyed. As a source of blood-forming stem cells, the animals obtained IV bone marrow cells of the same inbred strain. In addition, they obtained in experiment 5·10⁶ CD4 T cells and in experiment 2 5·10⁶ T cells (CD4 and CD8) of the congenic inbred strain PVG RT7^{b}. These animals are identical to the receiver strain PVG, except for the leukocyte surface molecule RT7 from which they express the allele b rather than a. By means of RT7^{b}-specific fluorescence-marked mAb, thus T cells can be identified in the animals originating from the 5·10⁶ injected mature T cells. Such T lymphocytes newly maturing from the immature precursor cells of the injected bone marrow in the thymus of the receiver animals to T lymphocytes, do not react however with RT7^{b}-specific fluorescence-marked mAb. The count of 5·10⁶ T lymphocytes corresponds approx, to one thousandth of the count of mature T lymphocytes of an adult rat, so that there is a model of drastic T lymphopenia. For multiplying the injected T cells in the receiver animal, according to the shown treatment scheme, 1 mg of the mitogenic anti-CD28 mAb JJ316 is applied IV at two days (day 0 and day 10 in experiment 1, day 0 and day 13 in experiment 2). At the mentioned times, blood was taken from the animal, and the count of the T lymphocytes was determined by measurement of the total count of the leukocytes and of the share of T lymphocytes therein. Furthermore, a differentiation was made between T lymphocytes originating from the mature injected T cells (RT7^{b}-positive) and those being newly generated in the thymus (RT7^{b}-negative). These analyses were performed by means of monoclonal antibodies with specificity for T cell surface markers. The employed technology is the multi-color immunofluorescence in conjunction with the flow cytophotometry (“FACS” analysis).

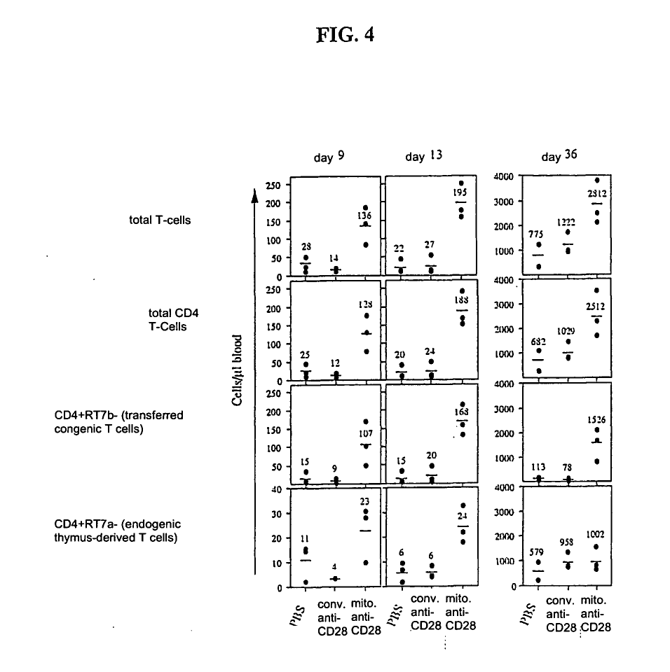

In FIG. 4 is shown the experiment 1, namely reconstitution with mature CD4 T cells. The different treatments of the animals are represented on the abscissa; each animal is symbolized by a dot. Already nine days after the beginning of the experiments, a clear lead in the count of T lymphocytes showed in the peripheral blood in the group treated with mitogenic anti-CD28 mAb over control animals which had obtained conventional anti-CD28 mAb or only the phosphate-buffered sodium chloride solution (PBS) used as a solvent for the mAb. This lead was kept till the last measuring point (day 36). If only the T lymphocytes originating from the mature RT7^{b}-positive CD4 T cells are considered, the therapeutic effect of the mitogenic anti-CD28 mAb is even more dramatic with regard to the control groups. The reason therefor is that with expiring time new RT7^{b}-negative T cells are generated in the thymus. The generation of these cells is independent from the antibody stimulation, was on the other hand however not affected thereby.

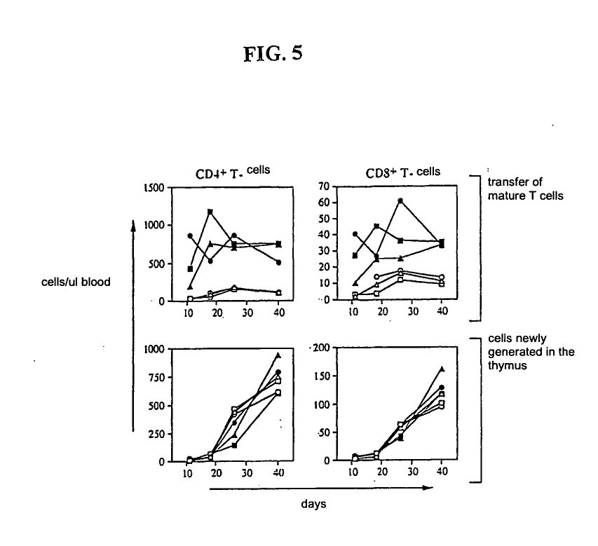

FIG. 5 shows the experiment 2, namely reconstitution with mature CD4 and CD8 T cells. Corresponding to the scheme in FIG. 3, 5·10⁶ RT7^{b}-positive T lymphocytes were transferred; approx. ¾ thereof are CD4, and approx. ¼CD8 T cells. The T cell counts in the blood were determined as described in FIG. 5. Each line represents a test animal. Open symbols indicate the treatments with PBS, full symbols that with the mitogenic anti-CD28 mAb JJ316. The upper two graphs show the development of the counts of RT7^{b}-positive cells deriving from the mature T cell inoculum; the lower two graphs show that of the RT7^{b}-negative cells newly generated in the thymus. The results show a quick multiplying of the mature CD4 and CD8 T lymphocytes by the treatment with mitogenic anti-CD28 mAb (top). The new generation of T cells (bottom) occurring at a later stage is not affected by the treatment.

### EXAMPLE 4

### Quality of the Multiplied T Lymphocytes

In this example is shown that T lymphocytes multiplied by the anti-CD28 therapy are durable and able to perform their function.

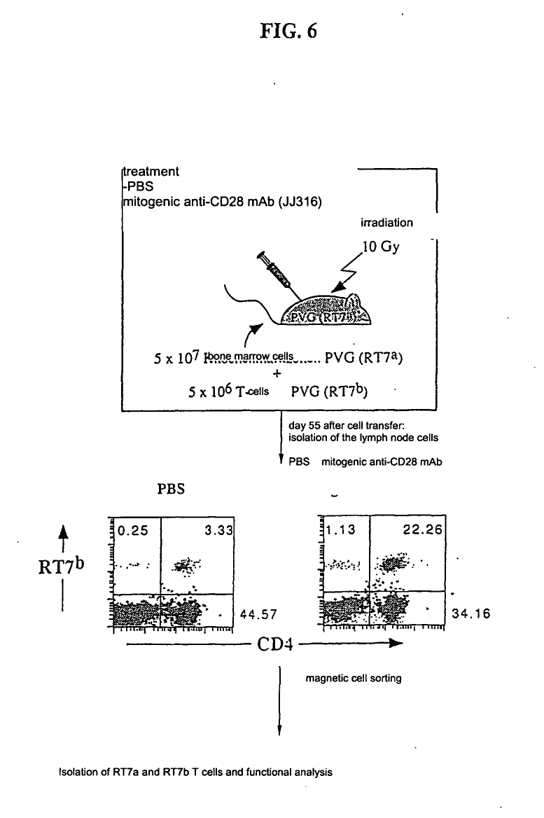

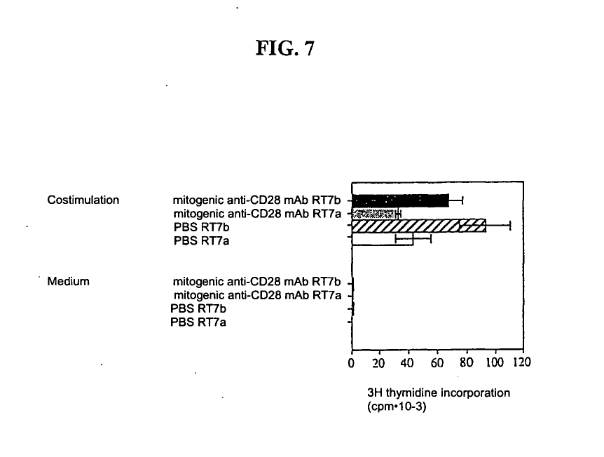

FIG. 6 shows the test plan and detection of T cells multiplied by anti-CD28 in the lymph nodes. The treatment of the test animals firstly corresponds to the scheme explained in FIG. 4, experiment 2. After 55 days the animals were killed. The analysis of the T lymphocytes in the lymph nodes had the result that after PBS treatment only 6% (3.33 of 47.9%) were derived from the inoculum of mature RT7^{b}-positive cells injected at the beginning of the test; the remaining CD4 T cells have been newly generated in the thymus (RT7^{b}-negative). In contrast thereto, the RT7^{b}-positive CD4 T cells in the treated group were approx. 40% (22.26 of 56.42%). The shown dotplots again show the results of the flow cytophotometric analyses. Fluorescence-marked antibodies against CD4 (abscissa) and RT7 (ordinate) were used. This result shows that the therapy made at the beginning of the test (day 0 and day 13) leads to a long-lasting repopulation with the T lymphocytes multiplied by the anti-CD28 therapy. RT7^{b}-positive and RT7^{b}-negative T lymphocytes were separated from one another. For this purpose, firstly an enrichment of the T cells by means of the passage over nylon wool was performed. Then the RT7^{b}-positive T cells are loaded with RT7^{b}-specific mAb to which were bound small paramagnetic iron balls. By means of a magnet, then the separation between RT7^{b}-positive and RT7^{b}-negative T cells was made. The system used here and being widely spread for the research of the company Miltenyi Biotech, Bergisch-Gladbach, Germany, is called MACS (magnetism activated cell sorting). The thus separated RT7^{b}-positive and RT7^{b}-negative T cells are then subjected to a functional analysis (FIGS. 7-9).

FIG. 7 shows the in vitro proliferation of in vivo therapeutically expanded T cells. The RT7^{b}-positive T cells isolated as described in FIG. 6, that is derived from the originally mature inoculum, and the RT7^{b}-negative (“RT7”-newly generated in the thymus) T cells were tested in a standard proliferation test for their reactivity against conventional costimulation. For this purpose, 1·10⁵ T cells were cultivated in 0.2 ml culture medium in 96-well microtiter plates for two days at 37° C., followed by a 16-hours pulse with 1 μCi3H-marked thymidine. The incorporation thereof in the DNA is represented as cpm×10⁻³ and is an accepted measure for the cell division activity (proliferation) of the cells. The upper four bars show approaches, wherein the T cells were stimulated by means of a T cell antigen receptor (TCR)-specific mAb (R73) and additionally with the conventional CD28-specific mAb JJ319 (classic costimulation). Here, the physiological signals for the T cell activation were imitated. The four bars show the reactions of the four different tested T cell populations, namely respectively the RT7^{b} originating from the original inoculum of mature T cells and the RT7^{a} newly generated in the thymus from both animal groups, namely the control animals treated with CD28 and with PBS. The lower group (“medium”) shows the same cells without stimulation. Here, no proliferation was observed. The test shows that even after the extensive in vivo expansion by CD28 therapy, the reaction capability of the T lymphocytes is maintained.

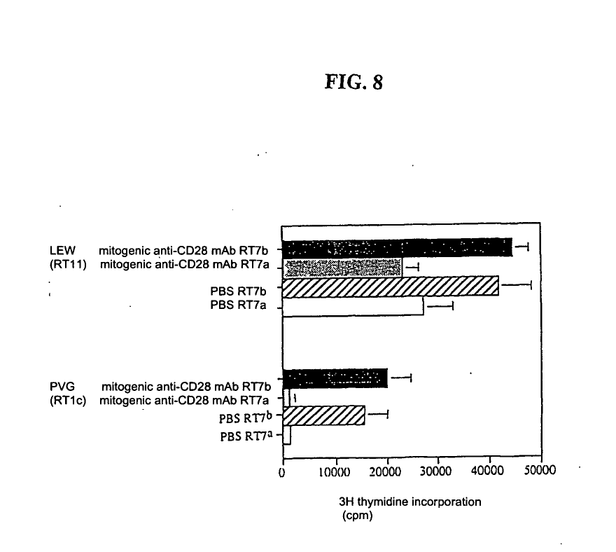

FIG. 8 shows that T cells multiplied in vivo by anti-CD28 therapy react against foreign transplantation antigens. The test approach basically corresponds to the one described in FIG. 7, with the difference that the stimulation of the T cells was not performed here by means of monoclonal antibodies, but by addition of so-called stimulator cells of the strain LEW. These are 10⁵ lymph node cells per well. The irradiation is performed, in order that the stimulator cells themselves cannot contribute to the measured proliferation. The strain LEW was selected, since it differs by its strong transplantation antigens (corresponding to HLA antigens of man) from PVG: LEW expresses RT1¹, PVG however RT1^{c}. For the determination of a base value, irradiated PVG lymph node cells were also used in the lower group. The result shows that again all of the four examined groups of T lymphocytes reacted with proliferation to the foreign transplantation antigens; furthermore an increased background reaction is found for the T cells of the treated as well as from the control group, said T cells originating from the originally applied inoculum of mature T cells (RT7^{b}-positive).

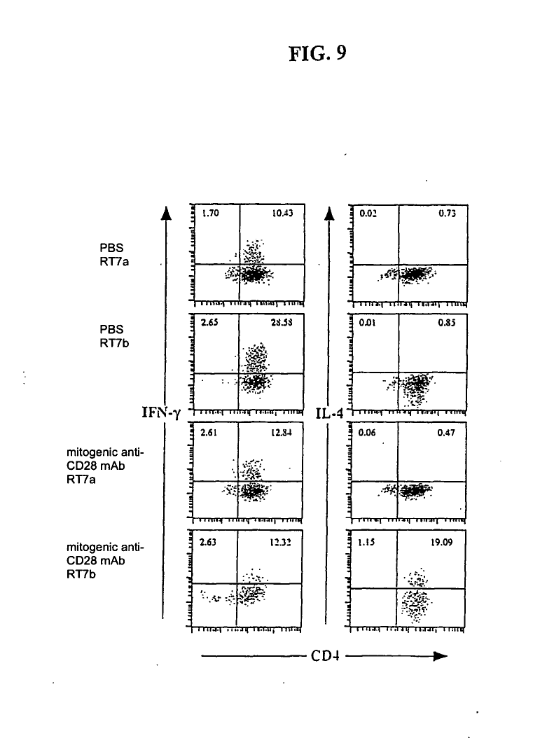

FIG. 9 shows that T cells in vivo multiplied by anti-CD28 therapy are able to produce cytokines. As described in FIG. 7, the expanded mature T cells (RT7^{b-}) and the T cells newly matured in the thymus (RT7^{b-}, corresponds to RT7^{a}) of the anti-CD28-treated and of the control animals are activated in vitro by costimulation. After 5 days, the cells were harvested and tested, by means of a protocol that can be found in the catalog of the company Pharmingen/Becton Dickinson, for the capability of producing the cytokines gamma-interferon and interleukin-4. These two cytokines were selected, since they are characteristic for the two functionally different types of CD4 effector T cells, which can be generated after activation: pro-inflammatory Th1 cells produce IFNgamma, anti-inflammatory and antibody production promoting Th2 cells produce IL-4. The detection method is an intracellular cytokine coloration being evaluated by flow cytophotometry. Each dot above the horizontally extending border line indicates a cell synthesizing the cytokine shown at the ordinate. It is obvious that all 4 groups of T lymphocytes contain a similarly large share of cells which can produce IFNgamma. In contrast thereto, IL-4 producing cells can only be found for those T lymphocytes which have been multiplied in vivo by mitogenic anti-CD28 therapy (dot plot bottom right). The experiment shows that by the in vivo expansion with mitogenic CD28-specific mAb, the capability of synthesizing cytokines of the Th1 type will not be lost. The occurrence of cells of the Th2 phenotype (producing W-4) is not surprising.

### EXAMPLE 5

### Multiplying T Cells by CD28 Therapy in Thymectomized Rats

The test approach corresponds to that in FIG. 3 with two differences: 1. The animals obtained one injection only of 1 mg mAb on day 0. 2. The thymus of the animals was removed by operation prior to irradiation and reconstitution, in or der to prevent further maturing of T cells in the thymus. Thereby, the situation of the lymphopenia for an adult man e.g. after a chemo or radiation therapy is imitated. Adult men are hardly capable for a new production of T cells in the thymus, since after the age of puberty the thymus function is practically discontinued.

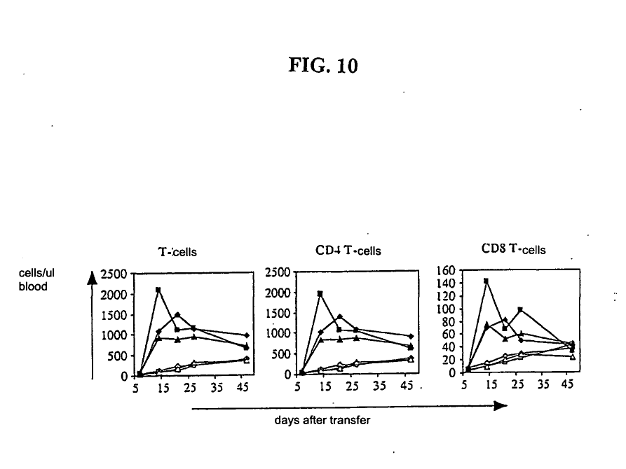

FIG. 10 shows the recovery of the T cell counts in the blood. A differentiation between RT7^{b}-positive and negative cells is now not necessary anymore, since all T cells are RT7^{b}-(lacking new production of RT7^{b} T cells in the thymus). The three graphs show the T cells and their CD4 and CD8 subpopulations of respectively three anti-CD28-treated animals (full symbols) and three animals treated with a control antibody of the same isotype (open symbols). As can easily be seen, the one-time treatment with 1 mg of the mitogenic anti-CD28 mAb JJ316 led to a very rapid recovery of the T cell counts.

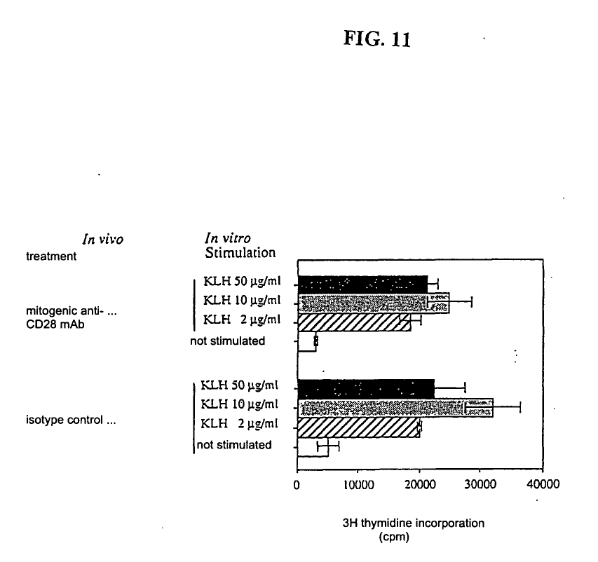

In FIG. 11 can be seen that by anti-CD28 therapy T cells multiplied in vivo can be immunized in vivo. The animals described in conjunction with FIG. 10 were immunized 4 weeks after the treatment with the model antigen keyhole limpet hemocyanin (KLH), in order to test the ability of the T lymphocytes to function as desired. After another 4 weeks the animals were killed, and the lymph node cells were tested in vitro for their ability to react with proliferation upon KLH stimulation. This is called the test for a “recall” antigen; such a test is used for instance for man in order to test the successful induction of a T cell immunity against vaccination antigens such as tetanus toxoid. The test system basically is equivalent to that of FIGS. 7 and 8, however the vaccination antigen KLH was used for the stimulation. It is obvious that the animals of both groups had a T cell immunity against KLH as a consequence of the vaccination. The T cell immunity is similarly strong in both groups, since the cell counts per culture well for the in vitro test are made alike, so that differences in the previous therapeutic T cell expansion by mitogenic antiCD28 mAb are eliminated.

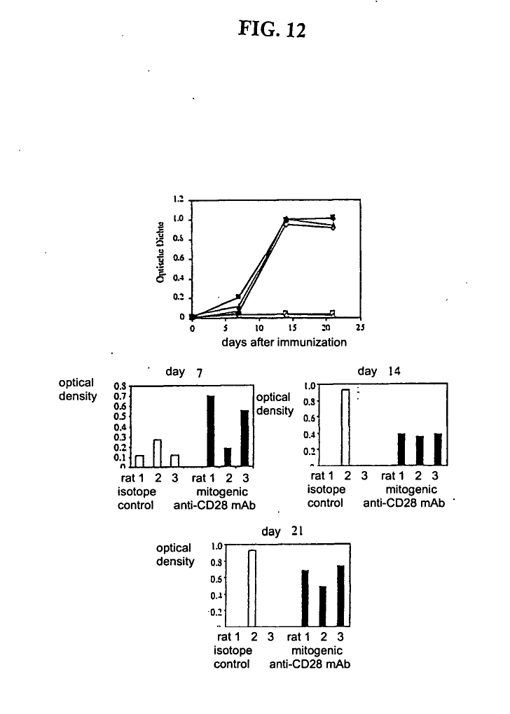

FIG. 12 shows the production of antibodies against the model antigen KLH. The production of antibodies by B lymphocytes depends on the function of CD4 T lymphocytes. Therefore, blood was taken at the times indicated on the abscissa from the KLH-immunized animals described in FIG. 11 and examined in an enzyme-linked immunosorbent assay (ELISA) for the presence of KLH-specific antibodies. As the upper graph shows, all anti-CD28-treated animals react with the same rapid kinetics on the immunization (full symbols), whereas two of the three control animals did not show a reaction; the third one reacted in a similar manner as the treated group, however with delayed kinetics (see the graphs of the ELISA results of the days 7, 14 and 21; these values were measured in a second test; the measured optical density is therefore not directly comparable to that of the upper graph).

In an experiment not represented in a figure it was shown that anti-CD28-treated T lymphopenic animals reject foreign skin transplants. The controlling of virus-infected cells, of tumors and of intracellular bacteria depends on the function of pro-inflammatory Th1 cells. Due to the presence of Th2 cells producing antiinflammatory IL-4 in CD28-treated T lymphopenic animals, the possibility had to be considered that the pro-inflammatory, “cell mediated” immunity is suppressed. This can be tested by the ability of the animals to reject a foreign skin transplant. This reaction, too, is Th1-dependent. The CD28-treated animals therefore obtained a skin transplant of the strain LEW expressing the RT1 allele of the strong transplantation antigens, whereas the treated PVG animals carry RT1. For verification, a syngenic PVG transplant was transferred. All of the in total eight examined treated animals rejected foreign skin, whereas the skin from a genetically identical strain could grow on. Exemplary were on one hand the necrotic LEW and on the other hand the well vascularized PVG transplant for an animal. On the average, the rejection by the treated animals happened in a shorter time than by the control animals. This result proves that by an anti-CD28 therapy the cell-mediated immunity is not lost.

### EXAMPLE 6

### Increasing the Granulocyte Counts in the Blood of Anti-CD28-Treated Rats

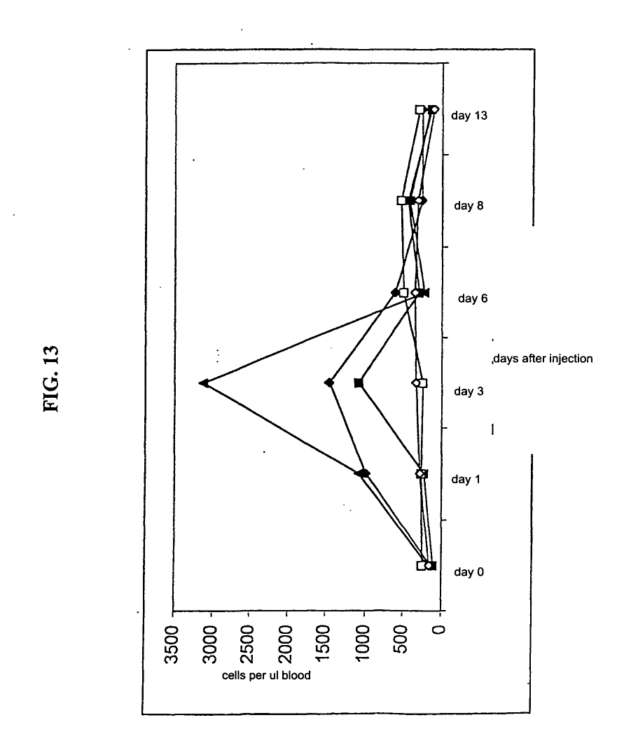

FIG. 13 shows the kinetics of the granulocyte count after treatment with conventional (open symbols) and with mitogenic anti-CD28 mAb (full symbols). Each line represents an animal. Adult LEW rats obtained 1 mg of the mitogenic anti-CD28 mAb JJ316 or of the conventional anti-CD28 mAb applied IV. The count of granulocytes in the peripheral blood was determined at the given times by determination of the total leukocyte count and of the share of the granulocytes therein. This measurement was made with the flow cytophotometry because of the special light scattering properties of granulocytes. As is shown in FIG. 13, in all animals which obtained mitogenic anti-CD28 mAb, a dramatic transient increase of the granulocyte count was observed, not however in the control animals which had obtained conventional anti-CD28 mAb.

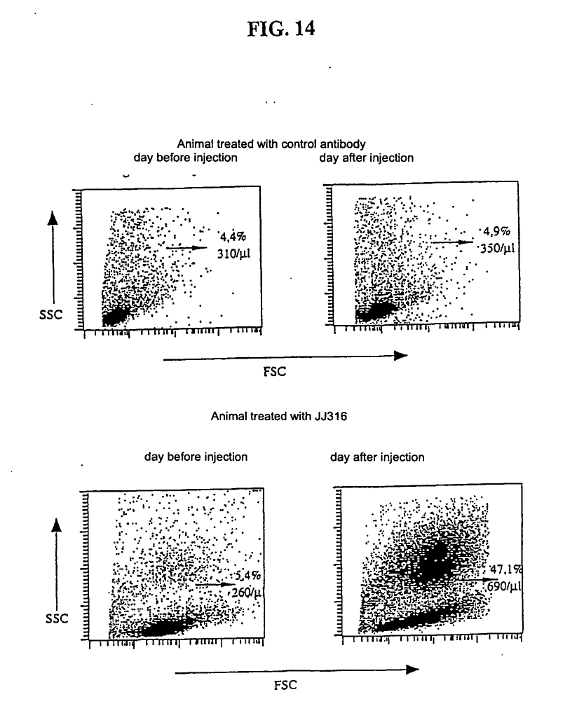

FIG. 14 shows an example for the increased granulocyte count in the blood of CD28-stimulated animals. The leukocytes were examined in a flow cytophotometer for their light scattering properties. FSC means forward scatter, SSC sideward scatter by 90° being a measure for the granularity of the cells. Every cell is represented by a dot. The text at the clouds indicates the granulocytes. The percentage is a direct result of the measurements, the cell count per μl blood is calculated by using the count of leukocytes previously determined per μl blood. It can be seen an increase of the granulocyte count by far exceeding the significance limit after the treatment with “direct” CD28-specific mAb.

### EXAMPLE 7

### Increasing the Monocyte Count After Treatment with Conventional and with Mitogenic Anti-CD28 mAb

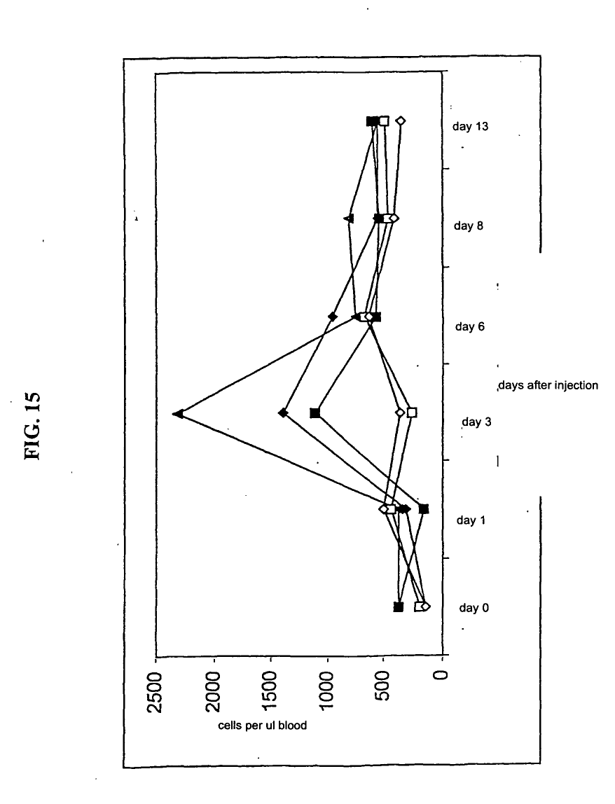

Adult LEW rats obtained 1 mg of the mitogenic anti-CD28 mAb JJ316 (full symbols) or of the conventional anti-CD28 mAb JJ319 (open symbols) applied IV. The count of the monocytes in the peripheral blood was determined at the given times by determination of the total leukocyte count and of the share of the monocytes therein. This measurement was made with the flow cytophotometry because of the special light scattering properties of monocytes and of the expression of the Mac-1 antigen detected by the fluorescencemarked mAb OX42. As is shown in FIG. 15, in all animals which obtained mitogenic anti-CD28 mAb, a dramatic transient increase of the monocyte count was observed, not however in the control animals which had obtained conventional anti-CD28 mAb.

### EXAMPLE 8

### Multiplying Granulocytes and Monocytes in the Blood of Irradiated Bone Marrow-Reconstituting Rats by Anti-CD28 Therapy

After impairment of the hematopoietic system for instance by radiation or chemotherapy, the granulocytes and monocytes attributed to the inherited or natural immune system must also be newly generated. Since activated T lymphocytes can stimulate them for instance by cytokine production (G-CSF, GM-CSF), and since in healthy animals an increase of the granulocyte and monocyte counts in the blood has been observed after anti-CD28 treatment, the capability of mitogenic anti-CD28 mAb to accelerate the recovery of this leukocyte populations was tested.

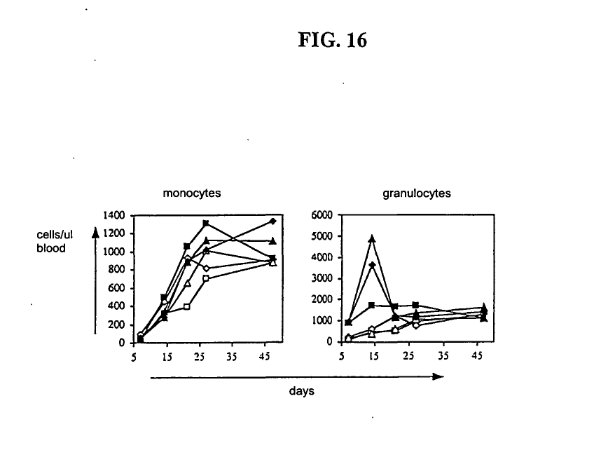

FIG. 16 shows the time dependence of granulocyte and monocyte counts in the blood of irradiated bone marrow-reconstituted rats after the anti-CD28 therapy.

Firstly, the thymus of PVG inbred rats was removed by operation, in order to prevent further maturing of T cells in the thymus (thereby, the situation of the lymphopenia for an adult man e.g. after a chemo or radiation therapy is imitated; for adult men, the thymus function is practically discontinued). They were then irradiated with gamma radiation of a cesium source so that the blood-forming system including the white blood cells is destroyed. As a source of blood-forming stem cells, the animals obtained IV bone marrow cells of the same inbred strain. In addition, they obtained 5·10⁶ CD4 T cells or 5·10⁶ T cells (CD4 and CD8) of the congenic inbred strain PVG RT7^{b}. The animals of this strain are identical to the receiver strain PVG, except for the leukocyte surface molecule RT7 from which they express the allele b rather than a. By means of RT7^{b}-specific fluorescence-marked mAb, thus T cells can be identified in the animals originating from the injected mature T cells. The count of the injected T cells corresponds approx, to one thousandth of the count of mature T cells of an adult rat, so that a model of drastic T lymphopenia exists. To the animals were applied at the day 0 1 mg JJ316 or JJ319, respectively. At the mentioned times, blood was taken, and the cell counts were determined according to example 7. From FIG. 16 can be taken a quicker recovery time of the monocyte and granulocyte count with the mAb according to the invention, in the case of the granulocytes in the first days after the treatment a highly accelerated reaction being found which then becomes normal again.

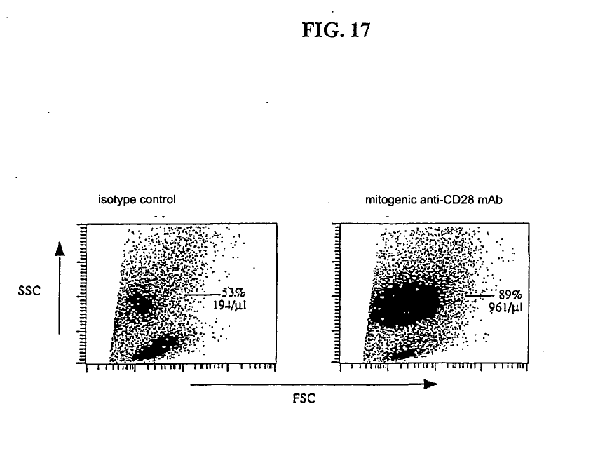

FIG. 17 shows data with regard to the share of granulocytes in the blood or irradiated bone marrow-reconstituted rats after anti-CD28 therapy. As an example, a measurement of the granulocytes share 7 days after the beginning of the therapy is shown. The method corresponds to the one described in example 3.

### EXAMPLE 9

### Stimulating in Rhesus Monkeys with a Mitogenic CD28-Specific Antibody

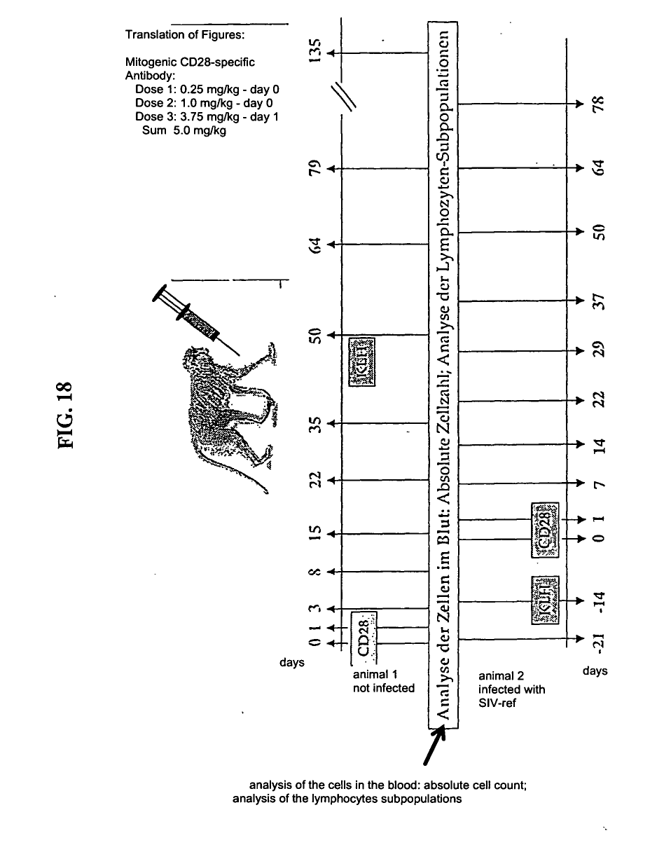

FIG. 18 shows the employed test plan. Two adult rhesus monkeys obtained IV a dose of 5 mg per kg body weight of a mitogenic CD28-specific mAb. In order to detect in time any occurring toxic effects, the dose was split up into three individual doses of 0.25, 1.0 und 3.75 mg/kg body weight, which were applied in the course of a day. Animal 1 was untreated, animal 2 had been infected 22 months before with an apathogenic virus (SIVΔ^{ref}). This infection does not play a role for the results shown here and is shown just to complete the picture. Animal 2 further was immunized 2 weeks before the antibody treatment with the model antigen KLH. None of the two animals showed any conspicuous reactions upon the antibody infusion. At the indicated times, blood was taken from the animals for the examinations described in the following.

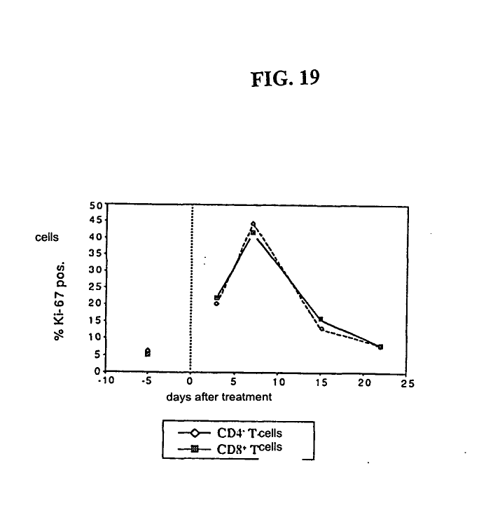

FIG. 19 shows the Ki-67 expression in T cells of a CD28-stimulated rhesus monkey (animal 2). The Ki-67 molecule was expressed in the nucleus of cells, which are in the cell cycle, i.e. in a stage of division. As the figure shows, prior to the treatment there were approx. 5% of the CD4 and 5% of the CD8 T cells in the cell cycle. After the CD28 treatment (day 0), the frequency first increased to 20%, then to 45% for both populations, and then dropped back again to 10%. This result shows that the CD28 stimulation in vivo stimulates the multiplying of the CD4 and CD8 T cells.

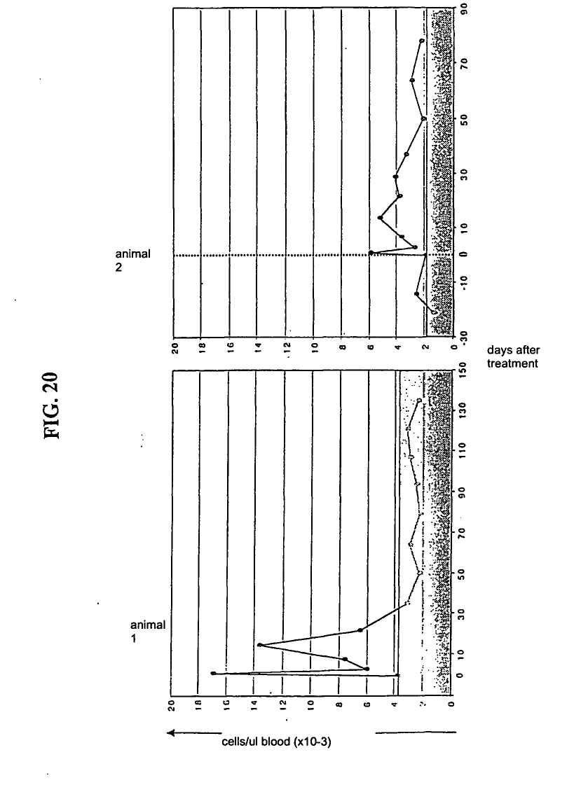

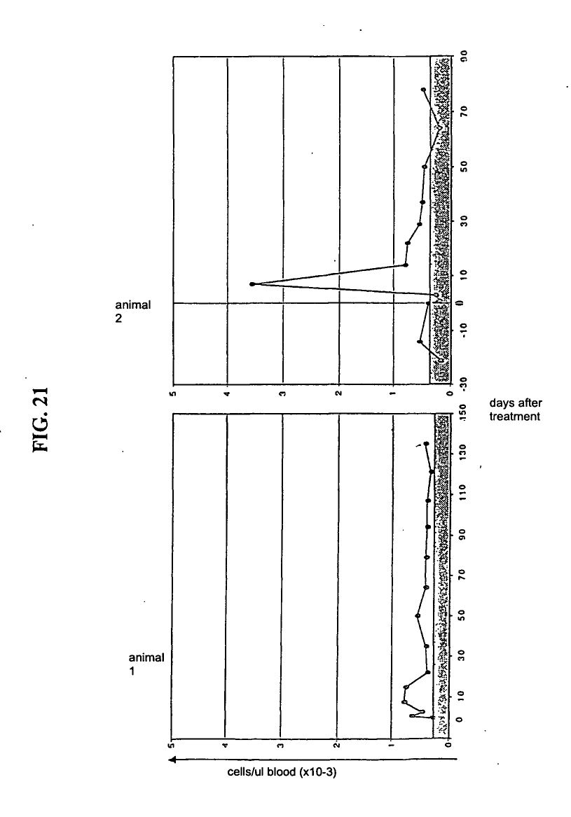

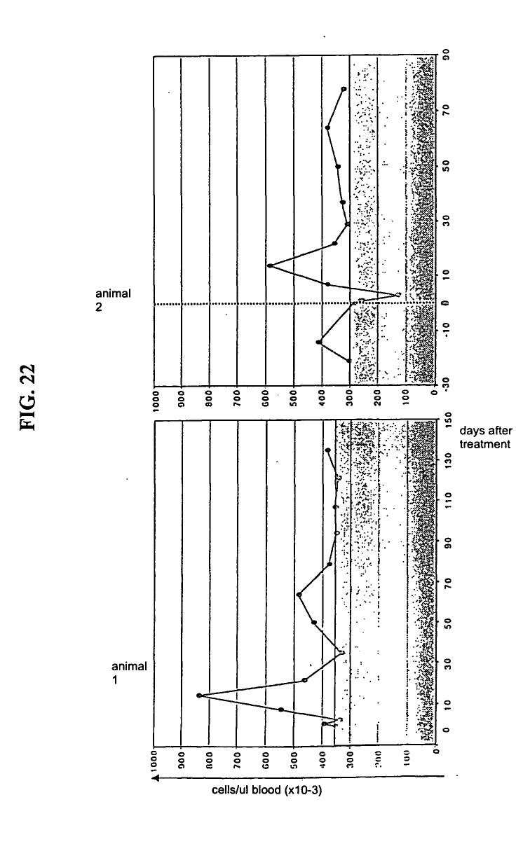

FIGS. 20 and 22 being essential for the invention finally show the obtainable increase of the count of granulocytes, monocytes and thrombocytes in the peripheral blood of anti-CD28-treated rhesus monkeys. The blood pictures were prepared in a hematology laboratory with a CellDyn 4000 device and are shown here as cells/μl blood as a function of time. FIG. 20 proves the increase of the count of neutrophilic granulocytes in the peripheral blood of anti-CD28-treated rhesus monkeys. Both animals showed a dramatic increase with regard to the original value. The latter was again reached for animal 1 after one month, for animal 2 slightly delayed. The therapy could make the usual treatment with G-CSF in the bone marrow transplantation unnecessary. FIG. 21 demonstrates the increase of the count of monocytes in the peripheral blood of anti-CD28-treated rhesus monkeys. In both animals, a clear increase over the original value with slow approximation to normal values during the following two months was observed. FIG. 22 finally shows the increase of the count of thrombocytes in the peripheral blood of anti-CD28-treated rhesus monkeys. Both animals showed a dramatic increase over the original value. This was reached again for animal 1 after one month, for animal 2 slightly earlier.

1. The use of monoclonal antibodies being specific for CD28 and activating T lymphocytes of several to all sub-groups without an occupation of an antigen receptor of the T lymphocytes and thus in an antigen-unspecific manner, or of an analogue hereto, for the preparation of a pharmaceutical composition for stimulating blood cells not carrying CD28.

2. The use of monoclonal antibodies being specific for CD28 and activating T lymphocytes of several to all sub-groups without an occupation of an antigen receptor of the T lymphocytes and thus in an antigen-unspecific manner, or of an analogue hereto, for the preparation of a pharmaceutical composition for treating diseases with a reduced number of blood cells not carrying CD28.

3. The use according to claim 1 or 2, wherein the blood cells are granulocytes.

4. The use according to claim 1 or 2, wherein the blood cells are monocytes.

5. The use according to claim 1 or 2, wherein the blood cells are thrombocytes.
